(19) United States
(12) Patent Application Publication
(10) Pub. No.: US 2013/0184805 A1
(43) Pub. Date: Jul. 18, 2013

(54) INTRODUCER SHEATH, PLACEMENT DEVICE FOR BLOOD VESSEL TREATMENT INSTRUMENT, AND METHOD FOR SHORTENING INTRODUCER SHEATH

(71) Applicant: TERUMO KABUSHIKI KAISHA, Tokyo (JP)

(72) Inventor: Akira SAWADA, Ashigarakami-gun (JP)

(73) Assignee: TERUMO KABUSHIKI KAISHA, Tokyo (JP)

(21) Appl. No.: 13/785,491

(22) Filed: Mar. 5, 2013

Related U.S. Application Data
(63) Continuation of application No. PCT/JP2011/065757, filed on Jul. 11, 2011.

(30) Foreign Application Priority Data
Sep. 28, 2010 (JP) 2010-217881

(51) Int. Cl. A61F 2/97

(52) U.S. Cl. CPC A61F 2/97
USPC 623/1.11

(57) ABSTRACT

A placement device for a blood vessel treatment instrument has an introducer sheath functioning as an outer tube, and also has an inner tube. The introducer sheath has a flexible tube-shaped sheath body and a hub into which the base end of the sheath body is inserted. The hub takes-up the base end of the sheath body into the hub by way of take-up shafts while slitting the base end of the sheath body by cutting blades, and thus the length of extension of the sheath body from the hub can be shortened.

This application is a continuation of International Application No. PCT/JP20011/065757 filed on Jul. 11, 2011 and claims priority to Japanese Application No. 2010-217881 filed on Sep. 28, 2010, the entire content of both of which is incorporated herein by reference.

### TECHNICAL FIELD

The disclosure here generally pertains to an introducer sheath and a placement device for a blood vessel treatment instrument. More specifically, the disclosure involves an introducer sheath whose overall length can be shortened, a placement device for a blood vessel treatment instrument, and a method for shortening an introducer sheath.

### BACKGROUND DISCUSSION

At the time of inserting a catheter into a blood vessel for diagnosis or treatment, in general, an introducer sheath is used as an access route which interconnects the exterior of the living body and the inside of the blood vessel. A primary purpose of this is to reduce the damage to a blood vessel which damage is generated, at the time of sequentially using a plurality of catheters, due to the process in which the catheters different in diameter are inserted into and withdrawn from a blood vessel puncture part a plurality of times. A secondary purpose is to ensure relatively easy insertion at the time of inserting a catheter having a non-smooth surface, such as a large-diameter balloon catheter or a balloon-expandable stent, and to avoid damage to a blood vessel.

On the other hand, in the case of a stent graft system used for treatment of aortic aneurysm (hereinafter referred also to simply as “system”), an introducer sheath is rarely used and the system is often inserted directly into a blood vessel. The stent graft system is a treatment device used in a stent graft insertion procedure in which a stent graft is carried to a treatment site where the aneurysm is present, then the stent graft is allowed to expand by its self-expanding force and is placed indwelling in situ. The stent graft system is by far larger than common catheters in shaft outside diameter, and this is why the introducer sheath is rarely used therewith.

A general stent graft system has an outer tube for housing a stent graft on the inner circumference of a distal portion thereof, and an inner tube slidably inserted inside the outer tube. The general stent graft system is so configured that with the outer tube slid proximally relative to the inner tube, the stent graft is allowed to expand radially by its self-expanding force, to be placed indwelling in a blood vessel. An example is disclosed in Japanese Patent Laid-open No. 2005-270395.

Most of the products of stent graft are of self-expanding type. As for the outer surface of the stent graft's shaft section to be inserted into the living body, in many cases, the stent part is also housed in a smooth tube, like in the cases of other self-expandable stents. Therefore, non-use of an introducer sheath at the time of inserting a stent graft system into a blood vessel would not produce the problem of damage to the blood vessel.

In the stent graft insertion procedure, however, there are cases where after the placement of a stent graft, another stent graft is added as an extension graft. There are also cases where a balloon catheter is inserted for the purpose of fixing the stent graft to the blood vessel more firmly. Therefore, there still remains the problem of damage to the blood vessel due to the repetition of insertion and withdrawal.

To address such a problem, there is a method in which the outer tube of the system is employed as an introducer sheath for other treatment device(s). In this method, after the first stent graft is inserted and placed indwelling, the outer tube (and the guide wire) of the system is left in the blood vessel, whereas the other component parts (inner tube, etc.) constituting the system are all pulled out. Then, the outer tube thus left in the blood vessel is used as an introducer sheath at the time of inserting other treatment device(s). This prevents spreading of the damage to the blood vessel puncture part at the time of inserting additional stent graft(s) or balloon catheter(s).

In most of the stent graft systems according to the art as above-described, it is necessary to insert the outer tube to the position (depth) where the stent graft is to be actually placed indwelling. Therefore, the overall length of the system inclusive of the outer tube is greater, as compared with common introducer sheaths. Therefore, in the case of using the outer tube of the system as it is as an introducer sheath at the time of inserting other treatment device, the treatment device must have an overall length not less than the overall length of the outer tube. Thus, treatment devices which are short in overall length cannot be used. In addition, a treatment device with an excessively large length is difficult to manipulate.

### SUMMARY

The disclosure here provides an introducer sheath which can be used as an introducer sheath for a short treatment device and which eliminates the need to use an excessively long treatment device, and also to provide a placement device for a blood vessel treatment instrument, and a method for shortening an introducer sheath.

The introducer sheath into which a long shaft is inserted, includes: a flexible tube-shaped sheath body; and a hub into which a proximal portion of the sheath body is inserted, wherein the hub takes-up a proximal portion of the sheath body into the hub while slitting the proximal portion of the sheath body, whereby the length of extension of the sheath body from the hub can be shortened.

It is possible, after the introducer sheath is employed as an outer tube of a blood vessel treatment instrument placement device to be used for placing a blood vessel treatment instrument (stent graft), to shorten the overall length of the sheath body of the introducer sheath. Therefore, a treatment device shorter than the overall length of the original introducer sheath can be inserted into a blood vessel by utilizing the introducer sheath. In addition, it is unnecessary to use an excessively long treatment device.

The hub preferably has a cutting section by which slits along an axial direction are formed in the sheath body; and a plurality of take-up shafts are preferably provided to respectively take-up terminal pieces of the sheath body separated by the slits.

By taking-up the terminal portions of the sheath body using the take-up shafts, it is possible to draw the sheath body into the hub and simultaneously to form slits in the sheath body by the cutting section. Therefore, take-up of the sheath body and formation of the slits are concurrently carried out by rotating the take-up shafts, so that the effective length of the sheath body can be easily shortened by a single operation (rotating operation).

The cutting section preferably forms the slits in portions of the sheath body which are on opposite sides with respect to a circumferential direction, and the plurality of take-up shafts are two take-up shafts disposed at positions spaced from each other along the direction of splitting of the sheath body by the slits.

The proximal portion of the sheath body is cut into two by the cutting section, and the resulting terminal ends are taken up by the two take-up shafts spaced from each other in the direction of splitting of the sheath body. Therefore, the sheath body can be rather smoothly taken up with a small number of component parts.

The cutting section preferably includes a plurality of cutting blades by which the slits are formed in circumferential-directionally different portions of the sheath body. The cutting resistance at the time of drawing the sheath body into the hub can thus be reduced. This helps ensure that the torque required at the time of taking up the terminal portions of the sheath body can be reduced, and the taking-up operation can be carried out more easily.

The hub preferably has an interlocking mechanism by which the plurality of take-up shafts are rotated in an interlocked manner. This allows all the take-up shafts to be rotated simultaneously, so that the separated terminal portions, resulting from cutting of the sheath hub, can be taken up in a well-balanced manner.

The hub preferably has rotation restraining mechanisms for inhibiting the take-up shafts from rotating in an unwinding direction. The take-up shafts are thus not rotated in the direction for unwinding the terminal portions of the sheath body having once been taken up, so that the sheath body is inhibited from moving in the extending direction relative to the hub. Therefore, the slits formed by the cutting section would not be exposed to the outside of the hub, so that blood can be prevented from leaking to the exterior of the hub through the slits.

The hub also preferably includes a hub body provided with a hollow section in which the take-up shafts and the proximal portion of the sheath body are housed. A rotational operating section which is rotationally operated from outside of the hub body can also be provided to thereby rotate the take-up shafts. The hub body is preferably configured to be liquid-tight so that a liquid flowing into the inside of the hub through the sheath body does not leak to the exterior. This helps ensure that even when blood flows into the hub through the sheath body, the blood is prevented from leaking to the exterior of the hub.

In accordance with the disclosure here, an introducer sheath into which is insertable an elongated shaft includes a hub possessing a distal end, a flexible tubular sheath body possessing a proximal-most portion and a distal-most portion; and movable shortening member mounted on the hub. The proximal-most portion of the sheath body includes a plurality of axially extending slits which circumferentially separate a plurality of terminal portions of the sheath body, with at least one of the terminal portions being connected to the shortening member, and the distal-most portion of the sheath body extending distally beyond the distal end of the hub. The shortening member is movable relative to the hub to move the sheath body relative to the hub and shorten an axial extent to which the distal-most portion of the sheath body extends distally beyond the distal end of the hub.

Another aspect of the disclosure here involves a blood vessel treatment instrument placement device by which a self-expanding blood vessel treatment instrument is delivered to and placed indwelling in a desired treatment site in a blood vessel. The blood vessel treatment instrument placement device comprises an introducer sheath having a sheath body for housing the blood vessel treatment instrument on an inner circumference of a distal portion of the sheath body, and an inner tube slidably positioned inside the sheath body. The introducer sheath comprises: a flexible tube-shaped sheath body possessing a proximal portion and a distal portion; and a hub in which the proximal portion of the sheath body is positioned with the distal portion of the sheath body extending distally beyond a distal end of the hub; with the hub being configured to take-up the proximal portion of the sheath body into the hub while slitting the proximal portion of the sheath body to shorten a length of the sheath body extending distally beyond the distal end of the hub.

After placing the blood vessel treatment instrument, it is possible, by pulling the inner tube out of the sheath body and shortening the effective length of the sheath body, to employ the introducer sheath as an introducer sheath for a treatment device which is shorter than the overall length of the original introducer sheath. In addition, it is unnecessary to use an excessively long treatment device.

Another aspect of the disclosure involves a method for shortening an introducer sheath in which a long shaft is inserted. The method includes slitting a proximal portion of a sheath body positioned in a hub to form at least two terminal portions at the proximal portion of a sheath body, drawing the sheath body into the hub in which the proximal portion of the sheath body is positioned, and taking up the terminal pieces of the sheath body.

### BRIEF DESCRIPTION OF DRAWINGS

FIG. 1 is a partly omitted side view of a blood vessel treatment instrument placement device according to an embodiment disclosed here.

FIG. 2 is a partly omitted perspective view of an introducer sheath shown in FIG. 1.

FIG. 3 is a partly omitted perspective view of the introducer sheath shown in FIG. 1.

FIG. 4 is a partly omitted schematic longitudinal cross-sectional view of the introducer sheath shown in FIG. 1.

FIG. 5 is a cross-sectional view taken along the section line V-V of FIG. 4.

FIG. 6 is a sectional view taken along the section line VI-VI of FIG. 4.

FIG. 7 is a transverse cross-sectional view of an introducer sheath according to a modification.

FIG. 8 is a partly omitted schematic perspective view of an introducer sheath according to another modification.

### DETAILED DESCRIPTION

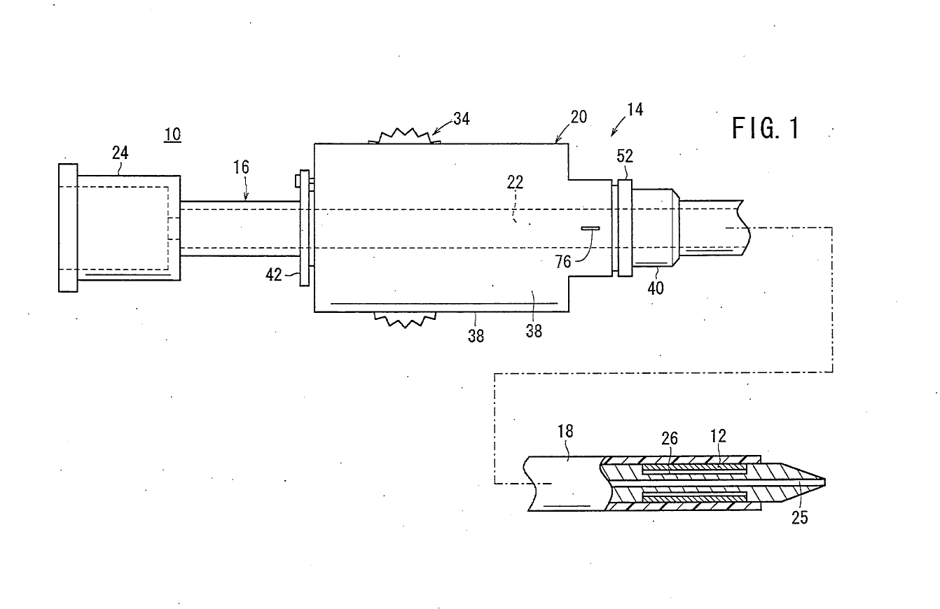

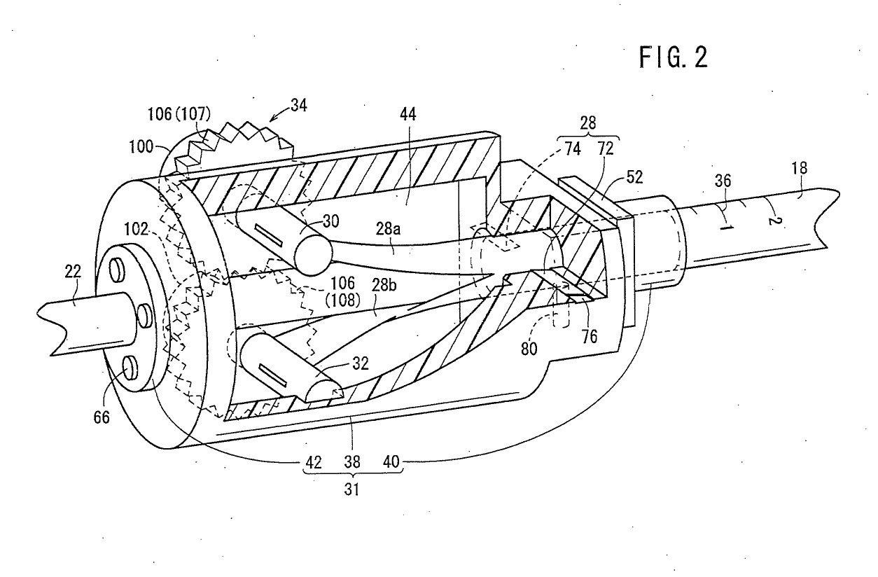

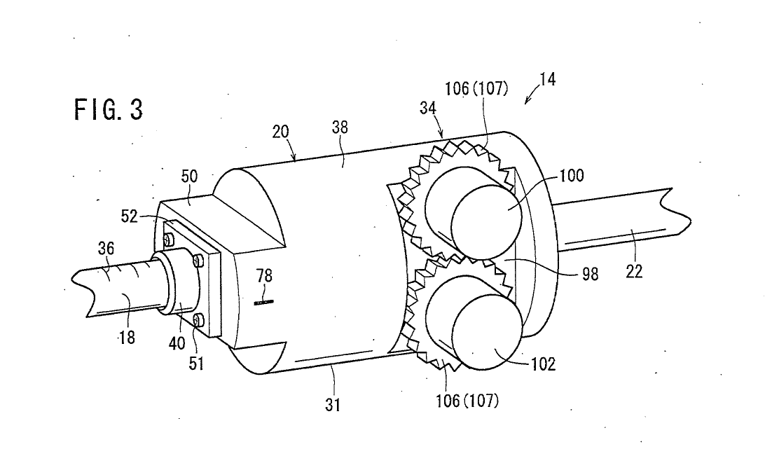

Set forth below is a description of an introducer sheath, a blood vessel treatment instrument placement device, and a method for shortening an introducer sheath according to the disclosure here. For convenience of description, the right side in FIGS. 1, 4 and 5 will be referred to as “distal end,” the left side in the FIGS. 1, 4 and 5 as “proximal end (rear end),” the right upper side in FIG. 2 as “distal end,” the left lower side in FIG. 2 as “proximal end (rear end),” the left lower side in FIG. 3 as “distal end,” and the right upper side in FIG. 3 as “proximal end (rear end).”

The blood vessel treatment instrument placement device 10 (hereinafter referred also to simply as “placement device 10”) shown in FIG. 1 is a device by which a stent graft 12 as a blood vessel treatment instrument having a self-expanding function is fed to and placed indwelling at a desired treatment site in a blood vessel. The stent graft 12 includes a stent formed in a tubular shape by knitting or braiding metallic wires, and a graft composed of a tubular cover disposed on the outer circumference of the stent. On one hand, the stent graft 12 can be contracted in diameter through elastic deformation by a radially inward force; on the other hand, the stent graft 12 has a self-expanding force for restoring its original size by an elastic force upon removal of the radially inward force. Thus, the stent graft 12 is configured as a self-expanding stent graft.

As shown in FIG. 1, the placement device 10 has an introducer sheath 14 and an inner tube 16. In performing a stent graft insertion procedure, the placement device 10 is used with the inner tube 16 inserted in the introducer sheath 14. The inner tube 16 is an example of an elongated shaft that is inserted into the introducer sheath 14.

The introducer sheath 14 has a sheath body 18 and a hub 20. The sheath body 18 is an outer tube which houses the stent graft 12 on an inner circumference of a distal portion of the outer tube. The sheath body 18 is formed from a flexible (elastic) material, and has a lumen having a substantially constant inside diameter along the axial direction of the sheath body. Examples of the material of the sheath body 18 include bio-compatible synthetic resins selected from among polyamides, polyethylene, fluoro-resins, polyimides and the like.

The hub 20 is a hollow member provided at (connected to) a proximal portion of the sheath body 18, and the hollow part of the hub 20 communicates with the lumen of the sheath body 18. The configuration of the hub 20 will be described in more detail below.

The inner tube 16 is inserted in the introducer sheath 14 in an axially slidable manner, and includes an inner tube body 22 and a hub 24. The inner tube body 22 is a flexible pipe-shaped body longer than the sheath body 18, and is provided therein along its axial direction with a guide wire lumen in which a guide wire is inserted. The inner tube body 22 has a maximum outside diameter not greater than the inside diameter of the sheath body 18 of the introducer sheath 14. Therefore, the inner tube body 22 can be pulled out of the sheath body 18 and the hub 20.

The inner tube body 22 is formed, in an outer circumferential portion near a distal portion of the inner tube body 22, with an annular groove 26 functioning as a housing section in which the stent graft 12 is housed or positioned. In FIG. 1, the stent graft 12 is covered by the sheath body 18 and, hence, inhibited from expansion. Examples of the material of the inner tube 16 include bio-compatible synthetic resins selected from among polyamides, polyethylene, fluoro-resins, polyimides and the like. The hub 20 is a hollow member provided at (connected to) a proximal portion of the inner tube 16.

In performing a stent graft insertion procedure by use of the placement device 10 configured as above, the placement device 10 in the state as shown in FIG. 1 is gradually inserted into a blood vessel along a guide wire precedently inserted in the blood vessel, to bring a distal portion of the placement device 10 to a desired treatment site. Upon the arrival of the distal portion at the desired treatment site, the introducer sheath 14 is moved (slid) in the proximal direction relative to the inner tube 16, so as to shift a distal portion of the sheath body 18 to the proximal side of the stent graft 12. This results in the stent graft 12, having been restrained by the inner circumferential surface of the sheath body 18 from expansion, expanding outwardly and pressing against the blood vessel under its self-expanding force. Consequently, the stent graft 12 is fixed and placed indwelling in the blood vessel.

The introducer sheath 14 is so configured that the inner tube 16 can be pulled out. Further, in order that the introducer sheath 14 is applicable also to short other treatment devices (for example, a balloon catheter), it is so configured that the effective length of the sheath body 18 (the length of extension of the sheath body 18 from the hub 20) can be shortened. In FIGS. 2 to 4, the sheath body 18 is in an initial state in which its effective length is not shortened.

As shown in FIG. 2, a proximal portion of the sheath body 18 is inserted into and fixed in the hub 20. The proximal portion of the sheath body 18 is slit or cut (divided, split or separated) into plural (in the example shown, two) pieces, and the terminal pieces 28a and 28b are each held or positioned inside the hub 20. The hub 20 holding the proximal portion of the sheath body 18 in such a state takes up the proximal portion of the sheath body 18 into the hub while slitting or cutting the proximal portion. The hub 20 includes a hub body 31, a cutting section 28, a plurality of take-up shafts 30, 32, and an operating section 34.

So that the amount by which the effective length of the sheath body 18 has been shortened can be seen, graduations 36 with numerals (numerical indicia) are provided on the outer circumferential surface of a proximal portion of the sheath body 18. The graduations, in cooperation with a distal member 40 which will be described later, indicate the length by which the sheath body 18 has been shortened. This point will be described later.

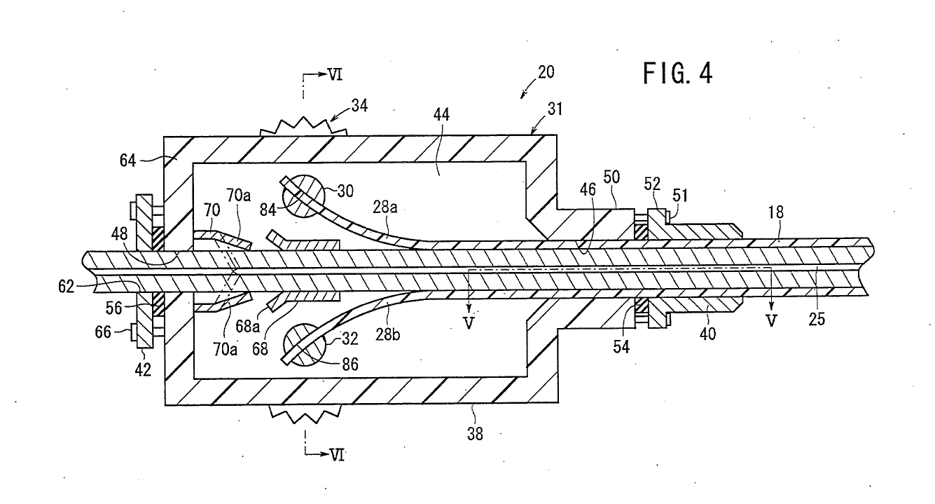

The hub body 31 includes a trunk member 38, the distal member 40, and a proximal member 42. The trunk member 38 has a hollow configuration. As shown in FIG. 4, the trunk member 38 is provided with a hollow section 44, a distal-side opening 46, and a proximal-side opening 48, and is further provided with a distal component section 50 on the distal side relative to the hollow section 44. Inside the hollow section 44, the proximal portion of the sheath body 18 is located, and the take-up shafts 30, 32 are disposed.

The distal-side opening 46 is a cylindrical hole which extends toward the distal side from the hollow section 44 and is approximately equal in diameter to the outer shape of the sheath body 18. The proximal side of the sheath body 18 is inserted through the distal-side opening 46. The proximal-side opening 48 is a cylindrical hole which extends toward the proximal side from the hollow section 44 and is approximately equal in diameter to the outer shape of the inner tube 16. The distal-side opening 46 and the proximal-side opening 48 are coaxial with each other, for permitting the inner tube 16 to be inserted through the distal-side opening 46 and the proximal-side opening 48.

The distal member 40 is a hollow cylindrical member having an inside diameter approximately equal to the diameter of the outer shape of the sheath body 18, and has a flange section 52 on the proximal side. The flange section 52 is fixed to the distal component section 50 by a fixing part or parts 51 such as a screw or screws.

The distal end face of the distal member 40 indicate the length by which the sheath body 18 has been shortened, in cooperation with the graduations 36 imparted to the outer circumferential surface of the sheath body 18. Specifically, when the sheath body 18 is in the initial state, a zero position of the graduations 36 (the graduation at the most proximal side) coincides with the distal end face of the distal member 40.

Between the flange section 52 of the distal member 40 and the distal component section 50, there is disposed a ring-shaped seal member 54 formed of an elastic material. An inner circumferential surface of the seal member 54 and an outer circumferential surface of the sheath body 18 are in secure contact with each other, over the whole circumference of the inner circumferential surface of the seal member 54 and the outer circumferential surface of the sheath body 18. This helps ensure liquid-tight sealing between the inner circumferential surface of the hub body 31 and the outer circumferential surface of the sheath body 18, at a distal-side portion of the hub body 31.

The material constituting the seal member 54 is not particularly restricted. Examples of the material which can be used include various rubbers such as natural rubber, butyl rubber, isoprene rubber, butadiene rubber, styrene-butadiene rubber, and silicone rubber. Incidentally, for seal members 56, 58 and 60 which will be described later, also, the materials mentioned above as material for the seal member 54 can be used.

The proximal member 42 is a circular disk-shaped member having an opening 62 approximately equal in diameter to the outer shape of the inner tube 16. The proximal member 42 is fixed to a rear wall 64 of the trunk member 38 by a fixing part or parts 66 such as a screw or screws. Between the proximal member 42 and the rear wall 64 is disposed a ring-shaped seal member 56 which is formed from an elastic material. An inner circumferential surface of the seal member 56 and an outer circumferential surface of the inner tube 16 are in secure contact with each other, over the whole circumference of the inner circumferential surface of the seal member 56 and the outer circumferential surface of the inner tube 16. This helps ensure liquid-tight sealing between the inner circumferential surface of the hub body 31 and the outer circumferential surface of the inner tube 16, at a proximal-side portion of the hub 31.

In order that another treatment device is smoothly guided to the distal-side opening 46 at the time of inserting the treatment device from the proximal side, the trunk member 38 is provided with a tubular guide member 68 in the hollow section 44 of the trunk member 38. A proximal portion 68a of this guide member 68 is formed in a flare shape spreading toward the proximal side so that the distal end of the treatment device is smoothly inserted through the proximal portion 68a of this guide member 68. To simplify the illustration in FIG. 2, the guide member 68 is omitted.

When the inner tube 16 is pulled out of the introducer sheath 14 in the condition where the introducer sheath 14 is inserted in a blood vessel, blood flows into the hollow section 44 through the sheath body 18. In view of this, as shown in FIG. 4, a check valve 70 as a valve element for hemostasis is provided on the front side (distal side) of the proximal-side opening 48. The check valve 70 has a plurality of valve elements 70a divided in the circumferential direction. To simplify the illustration in FIG. 2, the check valve 70 is omitted.

In the condition where the inner tube body 22 is inserted, as indicated by solid lines in FIG. 4, the check valve 70 is in a valve-opened state in which the valve elements 70a are spread radially outward. In the condition where the inner tube body 22 is pulled out, as indicated by imaginary lines in FIG. 4, the check valve 70 is in a valve-closed state in which the valve elements 70a are displaced radially inward to be closed. The check valve 70 helps ensure that even when the inner tube 16 is pulled out of the introducer sheath 14 inserted in a blood vessel, blood can be prevented from leaking to the outside of the hub 20.

Materials for forming the trunk member 38, the distal member 40 and the proximal member 42 are not particularly limited. For example, various metallic materials and various plastics and the like can be used singly or in combination.

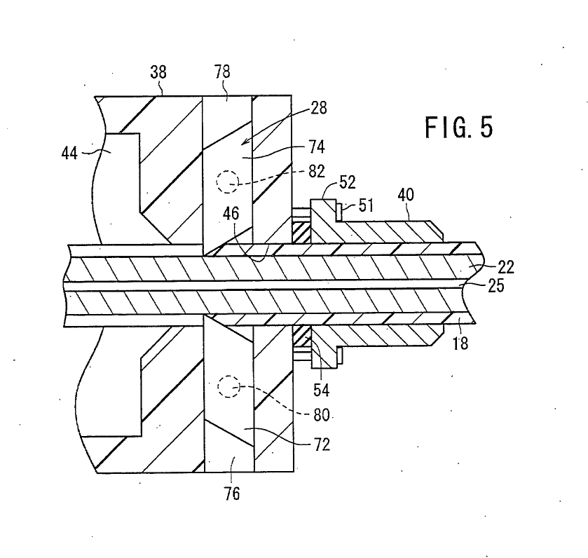

As shown in FIGS. 2 and 5, the cutting section 28 is disposed in the distal component section 50 constituting a portion on the distal side of the hollow section 44, in the trunk member 38. The cutting section 28 is a section by which slits along the axial direction (axially extending slits) are formed in the sheath body 18. The cutting section 28 in the example shown is so configured as to form slits in portions of the sheath body 18 which are located circumferentially or diametrically opposite to each other, namely, at portions deviated from each other by 180 degrees in circumferential phase. Specifically, the cutting section 28 has plural (in the example shown, two) cutting blades 72, 74.

The two cutting blades 72, 74 are disposed at positions between which the sheath body 18 interposed, with their cutting edges oriented toward the side (outer surface) of the sheath body 18. The cutting edges cut slits along the axial direction (axially extending slits) in the sheath body 18. The material constituting the cutting blades 72, 74 is not specifically restricted; for example, various metallic materials (stainless steel, etc.) can be used. With the cutting blades 72, 74 formed from a metallic material, it is easy to form the cutting edges.

As shown in FIG. 5, the trunk member 38 (the distal component section 50) is formed with plural (two) through-holes 76 and 78, each of which has its inner end opened at the inner circumferential surface of the distal-side opening 46 and has its outer end opened at the outer surface of the trunk member 38. The through-holes 76 and 78 function as slots in which the cutting blades 72, 74 are accommodated (disposed). The cutting blades 72, 74 are inserted from the outer ends of the through-holes 76, 78, and are positioned and fixed in the through-holes 76, 78 by fixing pins 80, 82.

The cutting edges of the cutting blades 72, 74 protrude from the inner ends of the through-holes 76, 78 (namely, from the inner surface of the distal-side opening 46) by an amount corresponding roughly to the material thickness (pipe wall thickness) of the sheath body 18, while also not making contact with the inner tube body 22. When the sheath body 18 is moved proximally, therefore, the sheath body 18 is cut by the cutting blades 72, 74 along the axial direction, but the inner tube body 22 is not cut.

Thus, the cutting blades 72, 74 are positioned by being inserted into and fixed in the through-holes 76, 78; therefore, the amounts of protrusion of the cutting edges from the inner circumferential surface of the distal-side opening 46 can be accurately set, and this state can be securely maintained. While each of the through-holes 76, 78 in the example shown extends in a direction orthogonal to the axis of the distal-side opening 46, it may extend in a direction inclined relative to the axis.

The configuration for disposing and fixing the cutting blades 72, 74 in the trunk member 38 is not restricted to the one shown in FIG. 5. For example, a configuration may be adopted wherein the hollow section 44 is extended toward the distal side, and a structure for disposing and fixing the cutting blades 72, 74 is provided in the extended part.

As shown in FIGS. 2 and 4, the terminal pieces 28a, 28b of the sheath body 18 which have been torn or slit (cut) are fixed respectively to the plural (two) take-up shafts 30, 32. The take-up shafts 30, 32 are members for respectively taking up the split or separated terminal pieces 28a, 28b of the sheath body 18 which have been slit or cut. The take-up shafts 30, 32 are disposed on the proximal side of the cutting section 28 in the hub body 31, and are rotatably supported on the trunk member 38. The take-up shafts 30, 32 are examples of movable shortening members configured to move relative to the hub 20 to shorten the extent to which the sheath body 18 (the distal-most portion of the sheath body 18) extends distally beyond the distal end of the hub 20.

The two take-up shafts 30, 32 in the example shown are arranged at positions spaced from each other along the direction of splitting or separation (the vertical direction in FIGS. 2 and 4) of the sheath body 18 by the slits. In addition, the two take-up shafts 30, 32 are located on opposite sides with reference to the axis of the hub 20 (the axis of the distal-side opening 46 and the proximal-side opening 48). Stated differently, with the introducer sheath 14 positioned horizontally so that the sheath body 18 is also positioned horizontally, the two take-up shafts 30, 32 are located on opposite sides of a horizontal plane in which the axis of the hub 20 lies. Furthermore, the axes (rotational center lines) of the two take-up shafts 30, 32 are parallel to each other.

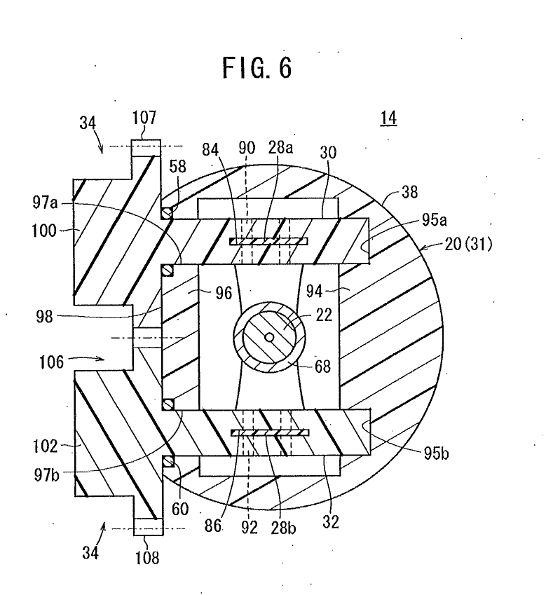

As shown in FIG. 6, the take-up shafts 30, 32 are provided with holes 84, 86 extending along the axial direction and penetrating in a diametrical direction. The terminal pieces 28a, 28b of the sheath body 18 are inserted into or positioned in the holes 84, 86, and are fixed in situ by a plurality of fixing pins 90, 92 arranged along the axial direction of the take-up shafts 30, 32. The fixation of the terminal pieces 28a, 28b of the sheath body 18 to the take-up shafts 30, 32 may be performed by other mechanisms or methods such as adhesion or fusion bonding.

As shown in FIG. 6, one-side end portions of the take-up shafts 30, 32 are respectively positioned in and supported by bottomed cylindrical holes 95a, 95b provided in a one-side wall 94 of the trunk member 38 of the hub body 31. Other-side end portions of the take-up shafts 30, 32 penetrate respective holes 97a, 97b provided in another-side wall 96 of the trunk member 38. Between the take-up shafts 30, 32 and the holes 97a, 97b are disposed seal members 58, 60 formed of an elastic material, whereby liquid-tight sealing is made between the take-up shafts 30, 32 and the holes 97a, 97b.

In addition, at the other-side end portions of the take-up shafts 30, 32, an operating section 34 to be operated to rotate the take-up shafts 30, 32 is provided outside of the hub body 31. In the configuration shown, the operating section 34 includes two operating wheels 100 and 102 which can be rotationally operated. As shown in FIGS. 3 and 6, the side portion of the hub body 31 is provided with a recess 98 having a bottom (flat bottom), and the operating wheels 100, 102 are disposed in the recess 98.

In this embodiment, the hub 20 further has an interlocking mechanism 106 by which rotations of the take-up shafts 30, 32 are interlocked with each other. In the configuration shown, the interlocking mechanism 106 includes intermeshing gears 107, 108 at outer circumferential portions of the operating wheels 100, 102. This ensures that when one of the two operating wheels 100, 102 is rotated, the other is rotated in the reverse direction.

The introducer sheath 14 configured as above-described can, after a stent graft insertion procedure, be used as an introducer sheath for other treatment device (for example, a balloon catheter) by a method in which the inner tube 16 is pulled off on the proximal side and then the effective length of the sheath body 18 is shortened. Before using the introducer sheath 14 disclosed here by way of example, the introducer sheath 14 is configured in the manner shown in FIGS. 1-6. That is, the proximal portion of the sheath body 18 possesses a plurality of slits so that the proximal portion of the sheath body 18 is split or divided to form the terminal pieces 28a, 28b that are positioned in the respective holes 84, 86 in the take-up shafts 30, 32 so that the terminal pieces 28a, 28b are secured to the take-up shafts 30, 32.

In order to shorten the effective length of the sheath body 18, it is necessary only to rotate the operating section 34. Specifically, when the operating wheels 100, 102 are rotated, the take-up shafts 30, 32 connected to the operating wheels 100, 102 rotate and the terminal pieces 28a, 28b are taken up on the take-up shafts 30, 32, simultaneously when the slits are formed by the two cutting blades 72, 74. That is, the cutting blades 72, 74 cut the sheath body 18 so that the axial extent of the slits, originally existing on the proximal portion of the sheath body 18 as shown in FIG. 2 prior to rotating the take-up shafts 30, 32 to shorten the sheath body 18, increases. As a result, the sheath body 18 is drawn into the hub 20, and the effective length of the sheath body 18 is shortened. The amount by which the sheath body 18 has been shortened can be confirmed by observing the graduations 36 (see FIGS. 2 and 3) on the outer circumferential surface of the sheath body 18.

As has been described above, the introducer sheath 14 ensures that after it is employed as an outer tube of the placement device 10 used for placement of the blood vessel treatment instrument (stent graft 12), the overall length of the sheath body 18 can be shortened. Therefore, a treatment device shorter than the overall length of the original introducer sheath 14 can be inserted into a blood vessel by utilizing the introducer sheath 14 shortened in effective length. In addition, it is unnecessary to employ a treatment device that is longer than needed.

According to the introducer sheath 14, it is possible, by taking up the terminal pieces 28a, 28b of the sheath body 18 by the plurality of take-up shafts 30, 32, to draw the sheath body 18 into the hub 20 and simultaneously form a cut or cuts in the sheath body 18 by the cutting section 28. Therefore, by rotating the take-up shafts 30, 32, take-up of the sheath body 18 and formation of the slits can be carried out concurrently. Accordingly, the effective length of the sheath body 18 can be relatively easily shortened by a single operation (rotating operation).

In addition, in the introducer sheath 14, a proximal portion of the sheath body 18 is torn up or split into two by the cutting section 28, and the terminal ends thus torn or split apart are taken up by the two take-up shafts 30, 32 spaced from each other in the direction of splitting of the sheath body 18. Therefore, the sheath body 18 can be relatively smoothly taken up by a small number of component parts.

The sharp cutting blades 72, 74 in the introducer sheath 14 are used as the cutting section 28, whereby the cutting resistance at the time of drawing the sheath body 18 into the hub 20 can be reduced. This helps make it possible to reduce the torque required for taking up the terminal pieces 28a, 28b of the sheath body 18, and to perform the taking-up (winding) operation more easily.

Furthermore, in the introducer sheath 14, the action of the interlocking mechanism 106 helps ensure that all the take-up shafts 30 and 32 can be simultaneously rotated by only rotationally operating either one of the operating wheels 100 and 102. Consequently the terminal pieces 28a and 28b torn apart can be taken up in a well-balanced manner. Accordingly, the sheath body 18 can be smoothly drawn into the inside of the hub body 31, and the taking-up operation can be carried out more easily.

In the introducer sheath 14, each part is sealed liquid-tight by the seal members 54, 56, 58, 60. Therefore, even when blood flows into the hub 20 through the sheath body 18, the blood is prevented from leaking to the exterior of the hub 20.

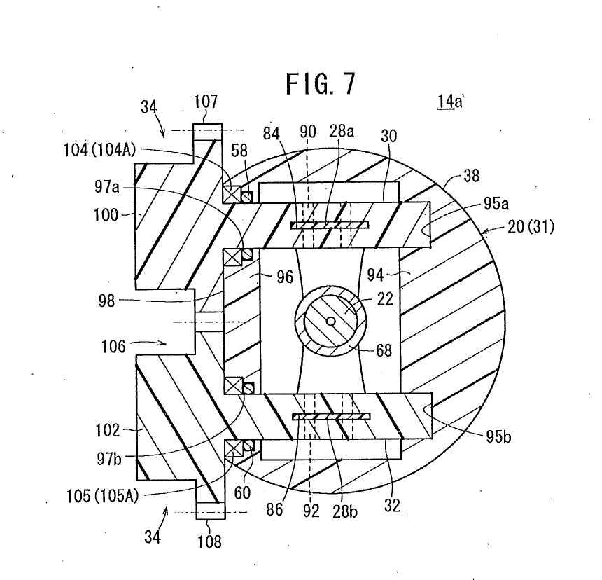

FIG. 7 illustrates, in transverse cross-section, a modified version of the introducer sheath 14a. This modified version of the introducer sheath 14a will be described below. The following description will focus primarily on the differences between this modified version of the introducer sheath 14a and the above-described introducer sheath 14. Features associated with the version of the introducer sheath 14a shown in FIG. 7 that are the same as features in the embodiment described above and shown in FIGS. 1-6 are identified by a common reference numerals and a detailed description of such features is not repeated.

The introducer sheath 14a shown in FIG. 7 has rotation restraining mechanisms 104, 105 for inhibiting the take-up shafts 30, 32 from rotating in an unwinding direction. In the configuration shown, the rotation restraining mechanisms 104, 105 are composed of one-way clutches 104A, 105A. Outer circumferential portions of the one-way clutches 104A, 105A are fixed to the trunk member 38 whereas inner circumferential portions are fixed to the take-up shafts 30, 32.

In the case of this embodiment, the take-up shafts 30, 32 are interlocked with each other through the gears 107, 108. Therefore, it is possible to employ a construction in which only one of the one-way clutches 104A, 105A is provided.

In the introducer sheath 14a configured as above-mentioned, the take-up shafts 30, 32 are inhibited by the rotation restraining mechanisms 104, 105 from rotating in the unwinding direction. This helps ensure that the terminal pieces 28a, 28b of the sheath body 18 which have once been taken up would not be unwound, so that the sheath body 18 is inhibited from moving in the extending direction (distal or forward direction) relative to the hub 20. Therefore, the slits formed by the cutting section 28 would not be exposed to the outside of the hub 20. Accordingly, blood can be prevented from leaking to the exterior of the hub 20 through the slits.

The configuration of the rotation restraining mechanisms 104, 105 is not restricted to the one-way clutches 104A, 105A. The configuration may be a ratchet mechanism having an engagement section for engagement with at least one of the gears 107, 108. The ratchet mechanism in this case is so configured that the engagement section is elastically displaced to ride over teeth of the gear(s) 107, 108 at the time of rotation in the winding direction of the take-up shafts 30, 32, whereas the engagement section does not ride over the teeth of the gear(s) 107, 108 at the time of rotation in the reverse direction to the winding direction. This configuration inhibits the take-up shafts 30, 32 from reverse rotation.

The introducer sheaths 14, 14a described above is configured so that the number of portions in which the slits are formed by the cutting section 28 is two, but the introducer sheaths are not limited in this regard. It is possible that the introducer sheaths 14, 14a can be configured so that the number of slits and the number of divided portions formed by the cutting section 28 is three or more. In other words, the proximal portion of the sheath body 18 may be taken up after being torn up or split into (divided into) three or more portions with three or more slits. Where the slits formed are three or more, the number of terminal pieces formed from the sheath body 18 is the same as the number of slits. In this case, a configuration may be adopted in which two take-up shafts 30, 32 are provided in the same manner as above, and two of the plural (three or more) terminal pieces are taken up by the take-up shafts 30, 32. Alternatively, a plurality of take-up shafts can be provided that are equal in number to the number of terminal pieces and the number of slits. In this case, the axes (rotational center lines) of the take-up shafts may not necessarily be parallel to one another.

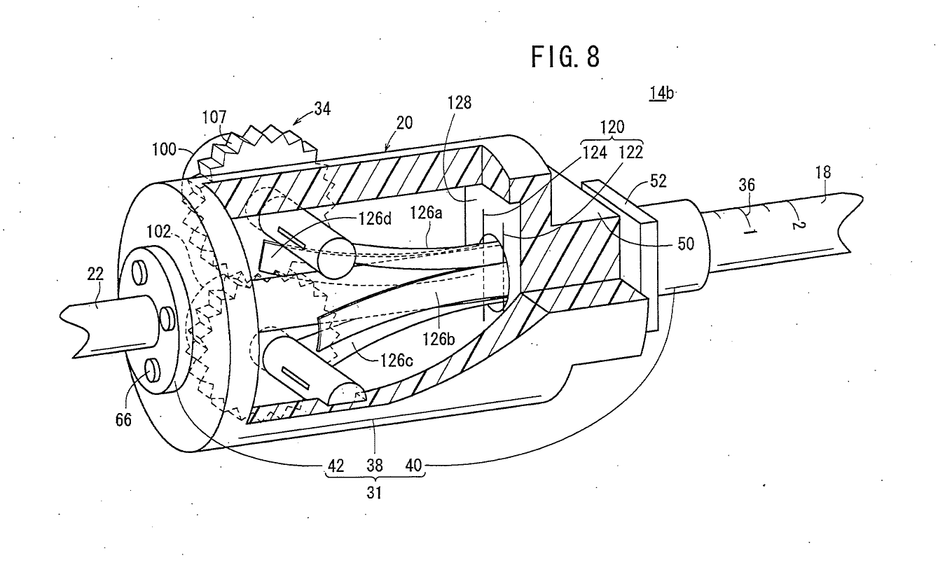

FIG. 8 illustrates, in a partly omitted perspective view, another modified version of an introducer sheath 14b. This modified version of the introducer sheath 14b will be described below. The following description will focus primarily on the differences between this modified version of the introducer sheath 14b and the above-described introducer sheath 14. Features associated with the version of the introducer sheath 14a shown in FIG. 8 that are the same as features in the embodiment described above and shown in FIGS. 1-6 are identified by a common reference numerals and a detailed description of such features is not repeated.

In the introducer sheath 14b shown in FIG. 8, a cutting section 120 for forming a plurality of slits in a sheath body 18 along the axial direction is composed of one or a plurality of linear members (in the example shown, two linear members 122, 124). The linear members 122, 124 may be, for example, wires, strings or the like.

In a hub body 31 in the example shown, two linear (filamentous) members 122, 124 are disposed substantially in parallel so that plural (four) slits are formed in the sheath body 18 on the side of the distal end of the hub body 31 relative to take-up shafts. Therefore, a proximal portion of the sheath body 18 is split into four terminal pieces 126a, 126b, 126c, 126d.

In the example shown, the two linear members 122, 124 are fixed to a distal-side wall surface 128 of a trunk member 38 in an appropriate manner, and are disposed to traverse a distal-side opening 46 (shown in FIG. 4). In place of the layout configuration shown, a configuration may be adopted in which the two linear members 122, 124 are embedded in a distal component section 50 so as to traverse the inside of the distal-side opening 46. The linear members 122, 124 are stretched with an appropriate tension exerted thereon so that the slits can be effectively formed in the sheath body 18 when the sheath body 18 is drawn into the hub 20. In order to reduce the cutting resistance at the time of forming the slits in the sheath body 18, it is preferable for the linear members 122, 124 to be sufficiently small in diameter within a range in which strength can be secured or assured.

In the condition where an inner tube body 22 is inserted in the sheath body 18, a part between both ends of each of the linear members 122, 124 is interposed between the sheath body 18 and the inner tube body 22. The two linear members 122, 124 are disposed at positions spaced from each other in the axial direction of the take-up shafts 30, 32 in such a manner as to extend in a direction substantially orthogonal to the axial direction of the take-up shafts 30, 32. Therefore, as shown in FIG. 8, two of the four terminal pieces 126a, 126b, 126c, 126b are split to both sides with respect to the spacing direction of the take-up shafts 30, 32, whereas the other two are split to both sides with respect to the axial direction of the take-up shafts 30, 32. The terminal pieces 126a, 126c are held by (fixed to) the take-up shafts 30, 32, respectively. The remaining terminal pieces 126b, 126d are left in the state of free ends, without being fixed to any other member or element in a hollow section 44. That is, the remaining terminal pieces 126b, 126d have free end portions that are cantilevered or unsupported.

In order to shorten the effective length of the sheath body 18 in the introducer sheath 14b, it suffices to rotate an operating section 34, like in the introducer sheath 14 shown in FIG. 2, etc. Specifically, when operating wheels 100, 102 are rotated, the take-up shafts 30, 32 connected to the operating wheels 100, 102 are rotated and the terminal pieces 126a, 126c are taken up by the take-up shafts 30, 32, simultaneously when the slits are formed in the sheath body 18 by the linear members 122, 124. As a result, the sheath body 18 is drawn into the hub 20, and the effective length of the sheath body 18 is shortened.

While the terminal pieces 126a, 126c are taken up by the take-up shafts 30, 32 as above-mentioned, the other terminal pieces 126b, 126d are not taken up. However, the terminal pieces 126b, 126d are flexible and can be rather easily deformed when an external force acts on the terminal pieces 126b, 126d. Therefore, the terminal pieces 126b, 126d are deformed inside the hollow section 44 upon making contact with an inner wall constituting the hollow section 44, during when the terminal pieces 126a, 126c are taken up by the take-up shafts 30, 32. Accordingly, take-up of the terminal pieces 126a, 126c by the take-up shafts 30, 32 would not be hampered.

In order to respectively take up the terminal pieces 126b, 126d, the hub body 31 may be further provided with other two take-up shafts. In this case, the take-up shafts for taking up the terminal pieces 126b, 126d are provided at positions which are on the side of the distal end or proximal end of the hub body 31 in relation to the take-up shafts 30, 32 and which are on opposite sides with reference to the axis of the hub 20 (the axis of the distal-side opening 46 and the proximal-side opening 48 (see FIG. 4)). In addition, the take-up shafts for taking up the terminal pieces 126b, 126d are rotatably provided on the trunk member 38, in such a manner that their axial direction is orthogonal to the axial direction of the take-up shafts 30, 32.

In FIG. 8, a configuration wherein the two linear members 122, 124 are provided has been shown as a configuration example of the cutting section 120. The cutting section 120 may have another configuration wherein a single linear member is disposed in a bent or crooked form such as to form four slits in the sheath body 18. Specifically, a configuration may be adopted wherein a part of a single linear member is disposed in the same manner as the linear member 122, and another part of the single linear member is disposed in the same manner as the linear member 124. This configuration ensures that four slits are formed in the sheath body 18 so that the sheath body 18 is split into the terminal pieces 126a, 126b, 126c, 126d.

The introducer sheath 14b shown in FIG. 8 may be provided with the rotation restraining mechanisms 104, 105 shown in FIG. 7.

The detailed description above describes features and aspects of embodiments, disclosed by way of example, of an introducer sheath, a placement device for a blood vessel treatment instrument, and a method for shortening an introducer sheath. But the invention is not limited to the precise embodiments and variations described and illustrated. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. An introducer sheath into which is insertable an elongated shaft, the introducer sheath comprising:
a hub possessing a distal end;
a flexible tubular sheath body possessing a proximal-most portion and a distal-most portion;
a movable shortening member mounted on the hub;
the proximal-most portion of the sheath body including a plurality of axially extending slits which circumferentially separate a plurality of terminal portions of the sheath body, at least one of the terminal portions being connected to the shortening member, and the distal-most portion of the sheath body extending distally beyond the distal end of the hub; and
the shortening member being movable relative to the hub to move the sheath body relative to the hub and shorten an axial extent to which the distal-most portion of the sheath body extends distally beyond the distal end of the hub.

2. The introducer sheath according to claim 1, wherein the proximal-most portion of the sheath body includes two slits which are diametrically opposed to each other so that the proximal-most portion of the sheath body includes two circumferentially spaced apart terminal pieces.

3. The introducer sheath according to claim 2, wherein the movable shortening member is a first movable shortening member, and including a second movable shortening member spaced apart from the first movable shortening member, the second movable shortening member being mounted on the hub and being movable relative to the hub.

4. The introducer sheath according to claim 1, wherein the shortening member is rotatably mounted on the hub, and one of the terminal portions of the proximal-most portion of the sheath body is wound on the rotatable shortening member as the shortening member is rotated.

5. The introducer sheath according to claim 1, further comprising a cutter positioned adjacent the proximal-most portion of the sheath body to contact and cut the proximal-most portion of the sheath body as the sheath body is moved relative to the hub to shorten the extent to which the distal-most portion of the sheath body extends distally beyond the distal end of the hub.

6. The introducer sheath according to claim 1, wherein the movable shortening member includes a plurality of take-up shafts each rotatably mounted on the hub, with each of the terminal pieces being connected to a respective one of the take-up shafts.

7. The introducer sheath according to claim 6, further comprising cutters mounted in the hub at positions to cut the sheath body and extend the slits;
The terminal portions comprising two terminal portions spaced apart from one another such that with the sheath body horizontally positioned, the two terminal portions are spaced apart from one another in a first direction and are positioned on opposite sides of a horizontal plane containing a central axis of the sheath body; and
the plurality of take-up shafts comprise two take-up shafts disposed at positions spaced from each other in the first direction.

8. The introducer sheath according to claim 7, wherein the cutters are spaced apart cutting blades each positioned adjacent the sheath body to contact and cut circumferentially spaced apart different portions of the sheath body.

9. The introducer sheath according to claim 6, wherein the hub comprises an interlocking mechanism by which the plurality of take-up shafts are rotationally interlocked so that rotation of one of the take-up shafts rotates another of the take-up shafts.

10. An introducer sheath into which is insertable an elongated shaft, the introducer sheath comprising:
a flexible tube-shaped sheath body possessing a proximal portion and a distal portion;
a hub in which the proximal portion of the sheath body is positioned with the distal portion of the sheath body extending distally beyond a distal end of the hub; and
the hub being configured to take-up the proximal portion of the sheath body into the hub while slitting the proximal portion of the sheath body to shorten a length of the sheath body extending distally beyond the distal end of the hub.

11. The introducer sheath according to claim 10, wherein the hub includes:
a cutting section to axially slit the proximal portion of the sheath body and separate the proximal-most portion of the sheath body into a plurality of terminal pieces; and
a plurality of take-up shafts, with one of the terminal pieces being connected to each of the take-up shafts.

12. The introducer sheath according to claim 11, wherein the cutting section is configured to form slits at circumferentially opposite locations on the proximal portion of the sheath body so that the terminal portions are spaced apart from one another in a first direction; and
the plurality of take-up shafts comprise two take-up shafts disposed at positions spaced from each other in the first direction.

13. The introducer sheath according to claim 11, wherein the cutting section comprises a plurality of spaced apart cutting blades each positioned adjacent the sheath body to contact and slit circumferential-directionally different portions of the sheath body.

14. The introducer sheath according to claim 11, wherein the hub comprises an interlocking mechanism by which the plurality of take-up shafts are rotationally interlocked so that rotation of one of the take-up shafts rotates another of the take-up shafts.

15. The introducer sheath according to claim 11, wherein the hub includes rotation restraining mechanisms that inhibit the take-up shafts from rotating in an unwinding direction.

16. The introducer sheath according to claim 11, wherein the hub comprises:
a hub body having a hollow section forming an inside in which the take-up shafts and the proximal portion of the sheath body are positioned;
a rotational operating section operatively connected to the take-up shafts and rotatably operable from outside the hub body to rotate the take-up shafts; and
the hub body being liquid-tight so that liquid flowing into the inside of the hub through the sheath body does not leak to outside the hub.

17. A blood vessel treatment instrument placement device by which a self-expanding blood vessel treatment instrument is delivered to and placed indwelling in a desired treatment site in a blood vessel, the blood vessel treatment instrument placement device comprising:
an introducer sheath having a sheath body for housing the blood vessel treatment instrument on an inner circumference of a distal portion of the sheath body;
an inner tube slidably positioned inside the sheath body;
the introducer sheath comprising:
a flexible tube-shaped sheath body possessing a proximal portion and a distal portion;
a hub in which the proximal portion of the sheath body is positioned with the distal portion of the sheath body extending distally beyond a distal end of the hub; and
the hub being configured to take-up the proximal portion of the sheath body into the hub while slitting the proximal portion of the sheath body to shorten a length of the sheath body extending distally beyond the distal end of the hub.

18. A method for shortening an introducer sheath in which an elongated shaft is inserted, the method comprising:
slitting a proximal portion of a sheath body positioned in a hub to form at least two terminal portions at the proximal portion of a sheath body;
drawing the sheath body into the hub in which the proximal portion of the sheath body is positioned; and
taking up the terminal pieces of the sheath body.

19. The method according to claim 18, wherein the sheath body is drawn into the hub and the terminal pieces are taken up by winding at least one of the terminal portions on a rotatable take-up roll.

20. The method according to claim 18, wherein the sheath body is drawn into the hub and the terminal pieces are taken up by winding two of the terminal portions on respective rotatable take-up rolls.
